(19) United States
(12) Patent Application Publication
(10) Pub. No.: US 2026/0166001 A1
(43) Pub. Date: Jun. 18, 2026

(54) PHYTOESTROGENIC FORMULATIONS FOR THE PROMOTION OF COGNITIVE FUNCTION, SLEEP QUALITY, AND MOOD SYMPTOMS

(71) Applicant: ARIZONA BOARD OF REGENTS ON BEHALF OF THE UNIVERSITY OF ARIZONA, Tucson, AZ (US)

(72) Inventor: Roberta Brinton, Tucson, AZ (US)

(21) Appl. No.: 19/127,899

(22) PCT Filed: Nov. 7, 2023
(86) PCT No.: PCT/US2023/078962

§ 371 Date: May 7, 2025

Related U.S. Application Data
(60) Provisional application No. 63/382,662, filed on Nov. 7, 2022.

(51) Int. Cl. A61K 31/353; A61P 25/20

(52) U.S. Cl. CPC A61K 31/353; A61P 25/20

(57) ABSTRACT

A method for promoting sleep quality in a subject is disclosed, involving the administration of an effective amount of a formulation comprising two or more phytoestrogen compounds or their analogues that selectively bind to estrogen receptor beta and cross the blood-brain barrier. The method is particularly applicable to menopausal or post-menopausal women, who may be identified as poor sleepers through actigraphy, a sleep questionnaire, or self-identification. The promotion of sleep quality includes improving sleep efficiency, reducing sleep latency, extending sleep duration, decreasing the number of awakenings, restoring the circadian rhythm of the chronobiology system, or a combination of these effects. The formulation may also include genistein, daidzein, or combinations thereof. Furthermore, the formulation can be administered orally, topically, transdermally, sublingually, or buccally, with effective dosages ranging from about 25 mg to 75 mg, and may be administered daily.

### CROSS-REFERENCES TO RELATED APPLICATIONS

This application claims benefit of U.S. Provisional Application No. 63/382,662 filed Nov. 7, 2022, the specification of which is incorporated herein in their entirety by reference.

### STATEMENT REGARDING FEDERALLY SPONSORED RESEARCH OR DEVELOPMENT

This invention was made with government support under Grant No. AG075122 awarded by National Institutes of Health. The government has certain rights in the invention.

### FIELD OF THE INVENTION

The present invention relates to phytoestrogenic (e.g., phytoSERM) formulations for the promotion of cognitive function, sleep quality, and mood symptoms.

### BACKGROUND OF THE INVENTION

Every year, approximately 1.5 million American women undergo the complex physiological changes associated with perimenopause, a distinctive neuroendocrine transition phase exclusive to the female population. Sleep disturbance is more common for women than men at any stage of their life. However, during the menopausal transition, the prevalence of sleep disturbance can increase dramatically (e.g., from 30% in premenopausal women to approximately 50% in peri- and postmenopausal women). Among menopausal symptoms, sleep disturbances are one of the most bothersome symptoms and are reported by 40% to 60% of menopausal women.

As of 2020, the United States is home to 45 million women aged 55 and older, while on a global scale, there are more than 850 million women between the ages of 40 and 60. This number is expected to exceed 1 billion by the year 2030. Recognizing the substantial and growing demographic of women experiencing these transformative life stages, the present invention seeks to address an unmet clinical need—promoting sleep quality in women during menopause.

### BRIEF SUMMARY OF THE INVENTION

It is an objective of the present invention to provide compositions and methods that enable the promotion of cognitive function, sleep quality, and mood symptoms, as specified in the independent claims. Embodiments of the invention are given in the dependent claims. Embodiments of the present invention can be freely combined with each other if they are not mutually exclusive.

Disclosed is a phytoestrogens (PhytoSERM) formulation that demonstrates agonist activity with selectivity for the estrogen receptor (ER) beta over ER alpha. Estrogen functions as a systems biology regulator in the brain to promote brain function. Midlife estrogen depletion is associated with multiple symptoms. An approximately equipotent mixture of genistein, daidzein, and S-equol, dosed orally at around 50 mg total (16.6 mg of each genistein, daidzein, and S-equol), selectively activates estrogen receptor beta (ERβ) to promote estrogenic action in the brain while inhibiting breast and uterus cell proliferation.

In some embodiments, the present invention features a method of promoting (e.g., improving) sleep quality in a subject. The method may comprise administering to the subject in need thereof an effective amount of a formulation comprising two or more phytoestrogen compounds or analogues thereof that selectively bind to estrogen receptor beta and cross the blood-brain barrier. In some embodiments, the subject is a menopausal or post-menopausal woman. In some embodiments, promoting sleep quality comprises increasing sleep efficiency, decreasing sleep latency, increasing sleep duration, decreasing the number of awakenings, or a combination thereof.

In other embodiments, the present invention features a method of improving sleep quality, cognitive function, or mood symptoms in a subject, the method comprising administering to the subject an effective amount of a formulation comprising two or more phytoestrogen compounds or analogues thereof that selectively bind to estrogen receptor beta and cross the blood-brain barrier. In some embodiments, the subject is a menopausal or post-menopausal woman. In some embodiments, promoting sleep quality comprises increasing sleep efficiency, decreasing sleep latency, increasing sleep duration, decreasing the number of awakenings, or a combination thereof.

In some embodiments, the subject has been identified as a poor sleeper. For example, the subject may be identified as a poor sleeper by actigraphy or a sleep questionnaire (e.g., a Pittsburgh Quality Sleep Index). Alternatively, the subject may self-identify as a poor sleeper. In certain embodiments, the subject is identified as having a menopause-related sleep disorder.

In some embodiments, one of the phytoestrogen compounds is S-equol. In some embodiments, the two or more phytoestrogen compounds may comprise genistein, daidzein, S-equol, or combinations thereof. The formulation may further comprise a pharmaceutically acceptable excipient or carrier. In some embodiments, the formulation may be administered orally, topically, transdermally, sublingually, or buccally. In some embodiments, the formulation is administered in an effective amount from about 25 mg to 75 mg. In other embodiments, the formulation is administered in an effective amount from about 50 mg to 75 mg. In certain embodiments, the formulation is administered in an effective amount of about 50 mg. Additionally, the formulation may be administered daily.

In some embodiments, the formulation is effective in restoring circadian rhythm of sleep. In some embodiments, promoting sleep quality improves cognitive function and/or improves mood symptoms.

In other embodiments, the present invention features a method of promoting sleep quality in a subject. The method comprises a) identifying a subject as having a menopause-related sleep disorder; and b) administering to the subject an effective amount of a formulation comprising two or more phytoestrogen compounds or analogues thereof that selectively bind to estrogen receptor beta and cross the blood-brain barrier; wherein the amount of the formulation is effective in treating the menopause-related sleep disorder.

One of the unique and inventive technical features of the present invention is the administration of a phytoestrogen formulation to individuals at risk for diminished sleep quality. Without wishing to limit the invention to any theory or mechanism, it is believed that the technical feature of the present invention advantageously provides for the promotion of quality sleep. Furthermore, the formulation of the present invention is designed for selectivity to the estrogen receptor beta (ERβ). This selectivity may also promote brain and breast health. None of the presently known prior references or work has the unique, inventive technical feature of the present invention.

Furthermore, the inventive technical features of the present invention contributed to a surprising result. For example, the formulations described herein comprise the combination of two or more phytoestrogens (e.g., three phytoestrogens) with high selectivity for estrogen receptor beta (ERβ). Selectively activating estrogen receptor beta (ERβ) promotes all of the estrogen outcomes that are critical for sustaining brain health and function, including sustaining glucose metabolism, mitochondrial function, synaptic plasticity, and reducing beta amyloid production in the brain, promoting health and vitality of the integumentary system and overall survival following loss of ovaries.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### DETAILED DESCRIPTION OF THE INVENTION

### Definitions

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

Unless otherwise defined herein, scientific and technical terms used in connection with the present application shall have the meanings that are commonly understood by those of ordinary skill in the art. Further, unless otherwise required by context, singular terms shall include pluralities, and plural terms shall include the singular. Thus, as used in this specification and the appended claims, the singular forms “a,” “an,” and “the” include plural referents unless the context clearly indicates otherwise.

Although methods and materials similar or equivalent to those described herein can be used to practice or test the disclosed technology, suitable methods and materials are described below. The materials, methods, and examples are illustrative only and not intended to be limiting.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder.

The terms “administering” and “administration” refer to methods of providing a composition or formulation to a subject. The compositions described herein can be administered in a number of ways depending on whether local or systemic treatment is desired and on the area to be treated. Such methods are well known to those skilled in the art and include, but are not limited to, administering the compositions orally, transdermally, topically, sublingually, or the like.

As used herein, the terms “co-administration” and “co-administering” refer to the administration of at least two agent(s) (e.g., phytoestrogen) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

Composition or formulation for oral administration include, but are not limited to, powders or granules, suspensions or solutions in water or non-aqueous media, pills, lozenges, capsules, sachets, or tablets. Thickeners, flavorings, diluents, emulsifiers, dispersing aids, or binders may be desirable. A person of skill monitoring a subject's clinical response can adjust the frequency of administration and dosage of the medication according to methods known in the art.

Composition or formulation for topical or transdermal administration may include ointments, lotions, creams, gels, drops, adherent patches, iontophoresis, suppositories, sprays, liquids, and powders. Conventional carriers, aqueous, powder or oily bases, thickeners, and the like may be necessary or desirable. A person of skill, monitoring a subject's clinical response can adjust the frequency of administration and dosage of the medication according to methods known in the art.

A composition can also be administered by buccal delivery or by sublingual delivery. As used herein, “buccal delivery” may refer to a method of administration in which the compound is delivered through the mucosal membranes lining the cheeks. In some embodiment, for a buccal delivery, the composition is placed between the gum and the cheek of a subject. As used herein, “sublingual delivery” may refer to a method of administration in which the compound is delivered through the mucosal membrane under the tongue. In some embodiments, for sublingual delivery, the composition is administered under the tongue of a subject.

An “acceptable carrier” refers to a non-toxic solid, semisolid, or liquid filler, diluent, encapsulating material, formulation auxiliary, or carrier conventional in the art for use with a therapeutic agent for administration to a subject. An acceptable carrier is non-toxic to recipients at the dosages and concentrations employed and is compatible with other ingredients of the formulation. The acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause an injection site reaction.

The term “effective amount” as used herein refers to the amount of a therapy that is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development, or onset of a given disease, disorder or condition, and/or to improve or enhance the prophylactic or therapeutic effect(s) of another therapy. In some embodiments, “effective amount,” as used herein, also refers to the amount of therapy provided herein to achieve a specified result.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular subject will depend upon a variety of factors, including the disorder being treated and the severity of the disorder; the specific composition employed; the age, body weight, general health, sex, and diet of the subject; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The exact amount of the compositions required may vary from subject to subject, depending on the species, age, weight, and general condition of the subject, the severity of the disorder being treated, the particular composition used, its mode of administration, and the like. Thus, it is not possible to specify an exact amount for every composition. However, an appropriate amount can be determined by one of ordinary skill in the art using only routine experimentation given the teachings herein.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the condition may be a menopause-related sleep disorder. For purposes of this invention, beneficial or desired clinical results include but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

As used herein, “clinical improvement” may refer to a noticeable reduction in the symptoms of a disorder, or cessation thereof.

As used herein, “cognitive function” may refer to the mental processes enabling the acquisition, processing, and use of information, including memory, attention, language, problem-solving, perception, learning, and reasoning. In certain aspects, e.g., the context of sleep, cognitive function may pertain to the mental processes and abilities influenced by sleep quality and quantity, including memory, attention, decision-making, problem-solving, and overall cognitive performance. Impaired sleep quality can detrimentally affect cognitive function and may lead to issues such as reduced alertness, memory difficulties, and compromised executive functioning.

As used herein, “mood symptoms” may refer to a range of emotional states and feelings, including happiness, sadness, anger, anxiety, depression, elation, irritability, apathy, contentment, and guilt, which are indicative of an individual's emotional well-being and can vary in intensity and duration. Sleep, in its role as described herein, influences mood symptoms by regulating emotions, reducing stress, enhancing mood, and mitigating conditions such as depression and anxiety. Adequate sleep contributes to better emotional regulation, reduces irritability, and fosters a positive feedback loop between improved mood and sleep quality.

“Estrogen Receptor,” as used herein, refers to any protein in the nuclear receptor gene family that binds estrogen, including, but not limited to, any isoforms and variants thereof. Human estrogen receptors include the alpha- and beta-isoforms (referred to herein as “ERα” and “ERβ”).

“Selective Estrogen Receptor Modulator” (or “SERM”), as used herein, refers to a compound that exhibits activity as an agonist or antagonist of an estrogen receptor (e.g., ERα, ERβ, or other estrogen receptor isoform) in a tissue-dependent or receptor dependent manner. Thus, as will be apparent to those of skill in the biochemistry, molecular biology, and endocrinology arts, compounds that function as SERMs can act as estrogen receptor agonists in some tissues, e.g., bone, brain, and/or cardiovascular, and as antagonists in other tissue types, e.g., the breast and/or uterine tissue.

“Phytoestrogen” refers to a naturally occurring compound of plants, such as soybeans, or plant products, such as whole grain cereals, that acts like estrogen or binds to an estrogen receptor.

As used herein, the term “PhytoSERM” refers to natural source compounds that preferentially target estrogen receptor beta.

As used herein, the term “analogue” refers to a chemical compound with a structure similar to that of another (reference compound) but differing from it in respect to a particular component, functional group, atom, etc.

As used herein, the term “derivative” refers to compounds which are formed from a parent compound by chemical reaction(s).

### Compositions and Methods of Use Thereof

The present invention may feature a method of promoting sleep quality (e.g., improving sleep quality) in a subject. The method may comprise administering to the subject in need thereof an effective amount of a formulation comprising two or more phytoestrogen compounds or analogues thereof that selectively bind to estrogen receptor beta and cross the blood-brain barrier. In other embodiments, the formulation comprises three or more phytoestrogen compounds or analogues thereof that selectively bind to estrogen receptor beta and cross the blood-brain barrier.

As used herein, “sleep quality” may refer to the overall effectiveness and restorative value of a subject's sleep. Sleep quality may be measured by various metrics including but not limited to sleep efficiency, sleep latency, sleep duration, and number of awakenings, e.g., after sleep onset. Sleep efficiency may refer to the ratio of total time spent asleep to time spent in bed. Sleep latency may refer to the amount of time it takes to fall asleep after getting in bed and turning off the lights. Sleep duration refers to the total amount of sleep obtained in a 24-hour period. In some embodiments, promoting sleep quality may further comprise restoration of the chronobiological architecture of sleep-e.g., normalized circadian rhythm.

In some embodiments, promoting sleep quality comprises increasing sleep efficiency (e.g., increasing time asleep), decreasing sleep latency (e.g., reducing time to fall asleep), increasing sleep duration (e.g., increasing time asleep), decreasing the number of awakenings (e.g., decreasing the number of times awake), or a combination thereof. In other embodiments, promoting sleep quality comprises increasing sleep duration (e.g., increasing time asleep), decreasing the number of awakenings (e.g., decreasing the number of times awake), normalizing circadian rhythms, or a combination thereof.

In other embodiments, promoting sleep quality comprises one or more of: decreasing nighttime awakenings, decreasing restlessness, decreasing time to fall asleep, increasing time asleep, increasing deep sleep, increasing rapid eye movement (REM) sleep, increasing restorative sleep, and improving sleep quality.

In some embodiments, promoting sleep may comprise increasing deep sleep, increasing rapid eye movement (REM) sleep, increasing restorative sleep, and increasing sleep quality. The formulations described herein may also promote sleep quality by reducing the incidence and/or severity of insomnia. In some embodiments, the formulation may be effective in reintroducing a normal circadian rhythm.

In some embodiments, the subject is a woman. In preferred embodiments, the subject may be a menopausal or post-menopausal woman. In other embodiments, the subject is a perimenopausal woman, e.g., a perimenopausal woman with menopausal symptoms such as hot flashes.

In some embodiments, the subject may have been identified as a poor sleeper. As non-limiting examples, actigraphy or a sleep questionnaire (e.g., a Pittsburgh Quality Sleep Index) may have been used to identify the subject as a poor sleeper. Optionally, the subject may self-report as a poor sleeper. In certain embodiments, the subject is identified as having a menopause-related sleep disorder.

In some embodiments, the formulation is effective in restoring circadian rhythm of sleep. In some embodiments, promoting sleep quality improves cognitive function and/or improves mood symptoms.

In some embodiments, the present invention features a method of promoting (e.g., improving) cognitive function, sleep quality, or mood symptoms in a subject. As a non-limiting example, the method may include administering to the subject an effective amount of a formulation comprising two or more phytoestrogen compounds or analogues thereof that selectively bind to estrogen receptor beta and cross the blood-brain barrier. In other embodiments, the formulation comprises three or more phytoestrogen compounds or analogues thereof that selectively bind to estrogen receptor beta and cross the blood-brain barrier.

In some embodiments, improving sleep quality comprises increasing sleep efficiency, decreasing sleep latency, increasing sleep duration, decreasing the number of awakenings, or a combination thereof. In some embodiments, improving cognitive function comprises increasing alertness and memory. In some embodiments, improving mood symptoms comprises reducing stress and enhancing mood.

In other embodiments, the present invention features a method of promoting sleep quality in a subject. The method may comprise identifying a subject as having a menopause-related sleep disorder and administering to the subject an effective amount of a formulation comprising two or more phytoestrogen compounds or analogues thereof that selectively bind to estrogen receptor beta and cross the blood-brain barrier. In preferred embodiments, the amount of the formulation is effective in treating the menopause-related sleep disorder. In some embodiments, the subject may be identified as having a menopause-related sleep disorder via actigraphy or a sleep questionnaire. As a non-limiting example, the sleep questionnaire may be the Pittsburgh Quality Sleep Index or any suitable alternative. The menopause-related sleep disorder may or may not result from vasomotor menopausal symptoms.

In some embodiments, the formulation comprises three or more phytoestrogen compounds or analogues thereof that selectively bind to estrogen receptor beta and cross the blood-brain barrier. In other embodiments, the formulation comprises three or more phytoestrogen compounds or analogues thereof that selectively bind to estrogen receptor beta and cross the blood-brain barrier. In some embodiments, the formulation comprises three phytoestrogen compounds or analogues thereof that selectively bind to estrogen receptor beta and cross the blood-brain barrier. In some embodiments, the formulation comprises two phytoestrogen compounds or analogues thereof that selectively bind to estrogen receptor beta and cross the blood-brain barrier. In other embodiments, the formulation comprises three or more phytoestrogen compounds or analogues thereof that selectively bind to estrogen receptor beta and cross the blood-brain barrier.

Phytoestrogen compounds described herein include estrogen-like compounds derived from plants. In some embodiments, the phytoestrogen compounds comprise isoflavones. In some embodiments, the phytoestrogen compounds comprise genistein, daidzein, equol, or combinations thereof. In other embodiments, the two or more phytoestrogen compounds may be selected from the group consisting of genistein, daidzein, equol, and combinations thereof.

In some embodiments, the phytoestrogen compounds comprise genistein, daidzein, S-equol, or combinations thereof. In other embodiments, the two or more phytoestrogen compounds may be selected from the group consisting of genistein, daidzein, S-equol, and combinations thereof.

Without wishing to limit the present invention to any theory or mechanism, it is believed that only having the S-isomer of equol increases the chemical purity of the formulations described herein.

In some embodiments, phytoestrogen analogues may include but are not limited to estrogen, 17 beta estrogen, or a combination thereof. In some embodiments, the phytoestrogen analogues may comprise an ERβ agonist.

In some embodiments, the phytoestrogen compounds may comprise genistein and daidzein. In other embodiments, the phytoestrogen compounds may comprise genistein and S-equol. In further embodiments, the phytoestrogen compounds may comprise S-equol and daidzein. In some embodiments, one of the phytoestrogen compounds may be S-equol. As a non-limiting example, the phytoestrogen compounds may comprise S-equol, genistein, and daidzein.

In some embodiments, the formulation comprising the selected phytoestrogen compounds may be administered in an effective amount from about 1 mg to about 10 mg, or about 1 mg to 25 mg, or about 1 mg to 50 mg, or about 1 mg to 75 mg, or about 1 mg to 100 mg, or about 1 mg to 125 mg, or about 10 mg to 25 mg, or about 10 mg to 50 mg, or about 10 mg to 75 mg, or about 10 mg to 100 mg, or about 10 mg to 125 mg. In some embodiments, the formulation comprising the selected phytoestrogen compounds may be administered in an effective amount from about 25 mg to 125 mg, or about 50 mg to 125 mg, or about 75 mg to 125 mg.

In some embodiments, the formulation comprising the selected phytoestrogen compounds may be administered in an effective amount from about 25 mg to 50 mg. In some embodiments, the formulation comprising the selected phytoestrogen compounds may be administered in an effective amount from about 25 mg to 75 mg. In some embodiments, the formulation comprising the selected phytoestrogen compounds may be administered in an effective amount from about 25 mg to 100 mg. In some embodiments, the formulation comprising the selected phytoestrogen compounds may be administered in an effective amount from about 50 mg to 75 mg. In some embodiments, the formulation comprising the selected phytoestrogen compounds may be administered in an effective amount from about 75 mg to 100 mg. In some embodiments, the formulation comprising the selected phytoestrogen compounds may be administered in an effective amount from about 50 mg to 100 mg.

In some embodiments, the formulation is administered in an effective amount of about 25 mg. In some embodiments, the formulation is administered in an effective amount of about 50 mg. In some embodiments, the formulation is administered in an effective amount of about 75 mg. In some embodiments, the formulation is administered in an effective amount of about 100 mg.

In some embodiments, the formulation may comprise equal amounts of the selected phytoestrogen compounds. For example, in a 50 mg dose of a formulation comprising three phytoestrogen compounds (e.g., genistein, daidzein, S-equol), there would be about 16.6 mg of each of the phytoestrogen compounds. Furthermore, as another example, in a 100 mg dose of a formulation comprising three phytoestrogen compounds (e.g., genistein, daidzein, S-equol), there would be about 33.3 mg of each of the phytoestrogen compounds. In other embodiments, the formulation may comprise unequal amounts of the selected phytoestrogen compounds.

In one non-limiting example, the formulation may comprise an oral dose of about 16.6 mg of each S-equol, genistein, and daidzein.

Without wishing to limit the present invention to any theory or mechanism, it is believed that a formulation comprising two or more phytoestrogen compounds is more effective than the same amount of individual phytoestrogen compounds.

The formulation may be administered once daily or twice daily (e.g., in divided doses). In some embodiments, the formulation may be administered at least once daily, at least once every other day, or at least once weekly or once monthly or administered for an extended period of time. In further embodiments, the formulation may be administered orally, topically, transdermally, sublingually, or through buccal delivery.

In some embodiments, the formulation may further comprise an acceptable excipient or carrier.

### EXAMPLE 1

The following is a non-limiting example of the present invention. It is to be understood that said example is not intended to limit the present invention in any way. Equivalents or substitutes are within the scope of the present invention.

Sleep disturbance is more common for women than men at any stage of their life. However, during the menopausal transition, the prevalence of sleep disturbance may increase dramatically (e.g., from 30% in premenopausal women to approximately 50% in peri- and postmenopausal women).

Among menopausal symptoms, sleep disturbances are one of the most bothersome symptoms and are reported by 40% to 60% of menopausal women. There is convincing evidence from several cross-sectional and longitudinal studies that the prevalence of perceived sleep disturbances increases in the menopausal transition, even after controlling for age. A recent meta-analysis of cross-sectional data from 24 studies reported higher odds of experiencing sleep disturbance in perimenopause (1.60), postmenopause (1.67), and surgical menopause (2.17) relative to women who are premenopausal.

The most common sleep-related complaint is nighttime awakenings. In a 7-year follow-up of 3045 women, the Study of Women's Health Across the Nation (SWAN) reported an increase in odds ratios (ORs) for difficulty staying asleep across the menopausal transition after adjusting for demographics and health-related factors. ORs also increased for difficulty falling asleep across the transition but decreased for early morning awakening from late perimenopause to postmenopause.

Although most studies examined associations between sleep and menopausal stages based on bleeding patterns, some studies have investigated the association between follicle-stimulating hormone (FSH) concentrations and self-reported sleep. Increasing FSH was associated with greater odds of waking up several times, whereas decreasing estrogen was associated with higher odds of difficulty falling and staying asleep in SWAN. Although epidemiologic studies show a clear increase in poor sleep quality as women enter menopause, the severity and persistence of poor sleep, as well as the extent of impairment in daytime function, varies between women.

Insomnia disorder is the most severe clinical manifestation of recurrent and chronic perceived poor sleep (difficulty falling asleep and staying asleep despite adequate opportunity to sleep), occurring three or more times per week and causing significant distress and daytime consequences. In the context of menopause, classic clinical features of insomnia, such as rumination, anxiety, and generalized hyperarousal, overlap with aspects of insomnia specific to menopause, such as vasomotor symptoms.

Higher sleep efficiency and shorter wakefulness after sleep onset (WASO) have been reported about ten weeks after starting hormone therapy (HT). Consistent findings have confirmed that women's sleep disturbances increase with age, more so than in men, and the prevalence of their sleep problems increases as they traverse the menopausal transition.

### Sleep Assessments

Sleep will be assessed using a combination of two approaches: (1) digital assessment of activity and sleep via actigraphy by wearable devices and (2) sleep questionnaire. These two approaches, in combination, will provide a multimodal, prospective assessment that will be able to capture habitual sleep-wake activity across 24 hours in free-living conditions.

Actigraphy-based sleep detection may be measured via an Empatica Embrace plus digital wristband. One of the innovative components of this example is incorporating a digital tool for assessing movement and sleep patterns as part of a strategy to determine changes in activity, such as daytime sleep and sleep integrity at night.

Actigraphy is a method of detecting activity and sleep by placing a small actigraph unit on the wrist for an extended period. The unit itself typically includes a small accelerometer and continually records the movements it undergoes. When the data are later uploaded to a computer, they can be analyzed and used to study circadian rhythm and wake-sleep patterns. It is useful for assessing daytime sleepiness in situations where a laboratory sleep latency test is not appropriate. Actigraphy is used to clinically evaluate insomnia, circadian rhythm sleep disorders, excessive sleepiness, and restless leg syndrome. It is also used in assessing the effectiveness of pharmacologic, behavioral, phototherapeutic, or chronotherapeutic treatments for these disorders. Actigraphy utilizes a portable device (actigraph) that records movement over extended periods of time and is typically worn on the wrist of the non-dominant arm. Sleep/wake patterns are estimated from periods of activity and inactivity based on this movement.

Actigraphy is based on the principle that there is reduced movement during sleep and increased movement during wake. Since its development in the early 1970s, actigraphy devices have become lighter, more durable, water resistant and have included features such as event markers and ambient light sensors. A modern actigraph uses accelerometers to detect wrist movement, which is sampled several times per second. These data are stored within the actigraph for up to several weeks.

The Pittsburgh Sleep Quality Index PSQI (Buysse, Reynolds, Monk, Berman, & Kupfer, 1989) is an effective instrument used to measure the quality patterns of sleep in older adults. It differentiates “poor” from “good” sleep by measuring seven domains: subjective sleep quality, sleep latency, sleep duration, habitual sleep efficiency, sleep disturbances, use of sleep medication, and daytime dysfunction over the last month. The client self-rates each of these seven areas of sleep. The scoring of the answers is based on a 0 to 3 scale, whereby 3 reflects the negative extreme on the Likert Scale. A global sum of “5” or greater indicates a “poor” sleeper.

Individual participant data will be listed by measure and time point for the following parameters: sleep duration, sleep fragmentation, sleep efficiency, sleep disturbance score, and sleep-related impairments score.

### Treatment Examples

Without wishing to limit the present invention to any particular theory or mechanism, it is believed that PhytoSERMs and formulations of the present invention may provide one or more of the following, alone or in combination: reduced incidence of sleep disturbance, reduced nighttime awakenings, reduced difficulty staying asleep, increased time asleep, reduced incidence of insomnia, reduced severity of insomnia, improve sleep quality, increased deep sleep, increased REM sleep, increased restorative sleep, reduced time to low heart rate, reduced restlessness, reduced Time to fall asleep, or reduced number of times awake. Restoration and sustaining of chronobiological/circadian sleep rhythm.

### EXAMPLE 2

The following is a non-limiting example of the present invention. It is to be understood that said example is not intended to limit the present invention in any way. Equivalents or substitutes are within the scope of the present invention.

A 48-year-old woman experiencing the challenges of menopausal sleep disturbances, characterized by difficulty falling asleep and maintaining uninterrupted sleep through the night, seeks a solution to improve her sleep quality. Thus, she purchases a once-daily pill comprising two or more phytoestrogen compounds (e.g., genistein, daidzein, S-equol) from a local store to improve her sleep quality. After one week of consistent use, she experiences quicker sleep onset and uninterrupted sleep throughout the night. These improvements are not only subjectively perceived but also objectively validated. No side effects are reported.

### EMBODIMENTS

The following embodiments are intended to be illustrative only and not to be limiting in any way.

Embodiment 1: A method of promoting sleep quality in a subject, the method comprising administering to the subject in need thereof an effective amount of a formulation comprising two or more phytoestrogen compounds or analogues thereof that selectively bind to estrogen receptor beta and cross the blood-brain barrier.

Embodiment 2: The method of embodiment 1, wherein the subject is a menopausal or post-menopausal woman.

Embodiment 3: The method of embodiment 1 or embodiment 2, wherein the subject has been identified as a poor sleeper.

Embodiment 4: The method of embodiment 3, wherein actigraphy or a sleep questionnaire has been used to identify the subject as a poor sleeper.

Embodiment 5: The method of embodiment 1 or embodiment 2, wherein the subject has been identified as having a menopause-related sleep disorder.

Embodiment 6: The method of embodiment 5, wherein actigraphy or a sleep questionnaire has been used to identify the menopause-related sleep disorder.

Embodiment 7: The method of embodiment 4 or embodiment 6, wherein the sleep questionnaire comprises the Pittsburgh Quality Sleep Index.

Embodiment 8: The method of any one of embodiments 1-7, wherein the subject has self-identified as a poor sleeper.

Embodiment 9: The method of any one of embodiments 1-8, wherein promoting sleep quality comprises increasing sleep efficiency, decreasing sleep latency, increasing sleep duration, decreasing the number of awakenings, or a combination thereof.

Embodiment 10: The method of any one of embodiments 1-9, wherein one of the phytoestrogen compounds is S-equol.

Embodiment 11: The method of any one of embodiments 1-10, wherein the two or more phytoestrogen compounds comprise genistein, daidzein, S-equol, or combinations thereof.

Embodiment 12: The method of any one of embodiments 1-11, wherein the formulation further comprises a pharmaceutically acceptable excipient or carrier.

Embodiment 13: The method of any one of embodiments 1-12, wherein the formulation is administered orally.

Embodiment 14: The method of any one of embodiments 1-12, wherein the formulation is administered topically or transdermally.

Embodiment 15: The method of any one of embodiments 1-12, wherein the formulation is administered sublingually or buccally.

Embodiment 16: The method of any one of embodiments 1-15, wherein the formulation is administered in an effective amount from about 25 mg to 75 mg.

Embodiment 17: The method of any one of embodiments 1-16, wherein the formulation is administered in an effective amount from about 50 mg to 75 mg.

Embodiment 18: The method of any one of embodiments 1-17, wherein the formulation is administered in an effective amount from about 50 mg.

Embodiment 19: The method of any one of embodiments 1-18, wherein the formulation is administered daily.

Embodiment 20: The method of any one of embodiments 1-19, wherein the formulation is effective in restoring circadian rhythm of sleep.

Embodiment 21: The method of any one of embodiments 1-19, wherein promoting sleep quality improves cognitive function.

Embodiment 22: The method of any one of embodiments 1-19, wherein promoting sleep quality improves mood symptoms.

Embodiment 23: A method of improving sleep quality, cognitive function, or mood symptoms in a subject, the method comprising administering to the subject an effective amount of a formulation comprising two or more phytoestrogen compounds or analogues thereof that selectively bind to estrogen receptor beta and cross the blood-brain barrier.

Embodiment 24: The method of embodiment 23, wherein the subject is a menopausal or post-menopausal woman.

Embodiment 25: The method of embodiment 23 or embodiment 24, wherein the subject has been identified as a poor sleeper.

Embodiment 26: The method of embodiment 25, wherein actigraphy or a sleep questionnaire has been used to identify the subject as a poor sleeper.

Embodiment 27: The method of embodiment 23 or embodiment 24, wherein the subject has been identified as having a menopause-related sleep disorder.

Embodiment 28: The method of embodiment 27, wherein actigraphy or a sleep questionnaire has been used to identify the menopause-related sleep disorder.

Embodiment 29: The method of embodiment 26 or embodiment 28, wherein the sleep questionnaire comprises the Pittsburgh Quality Sleep Index.

Embodiment 30: The method of any one of embodiments 23-29, wherein the subject has self-identified as a poor sleeper.

Embodiment 31: The method of any one of embodiments 23-30, wherein improving sleep quality comprises increasing sleep efficiency, decreasing sleep latency, increasing sleep duration, decreasing the number of awakenings, or a combination thereof.

Embodiment 32: The method of any one of embodiments 23-30, wherein improving cognitive function comprises increasing alertness and memory.

Embodiment 33: The method of any one of embodiments 23-30, wherein improving mood symptoms comprises reducing stress and enhancing mood.

Embodiment 34: The method of any one of embodiments 23-33, wherein one of the phytoestrogen compounds is S-equol.

Embodiment 35: The method of any one of embodiments 23-34, wherein the two or more phytoestrogen compounds comprise genistein, daidzein, S-equol, or combinations thereof.

Embodiment 36: The method of any one of embodiments 23-35, wherein the formulation further comprises a pharmaceutically acceptable excipient or carrier.

Embodiment 37: The method of any one of embodiments 23-36, wherein the formulation is administered orally.

Embodiment 38: The method of any one of embodiments 23-36, wherein the formulation is administered topically or transdermally.

Embodiment 39: The method of any one of embodiments 23-36, wherein the formulation is administered sublingually or buccally.

Embodiment 40: The method of any one of embodiments 23-39, wherein the formulation is administered in an effective amount from about 25 mg to 75 mg.

Embodiment 41: The method of any one of embodiments 23-40, wherein the formulation is administered in an effective amount from about 50 mg to 75 mg.

Embodiment 42: The method of any one of embodiments 23-41, wherein the formulation is administered in an effective amount from about 50 mg.

Embodiment 43: The method of any one of embodiments 23-42, wherein the formulation is administered daily.

Embodiment 44: The method of any one of embodiments 23-43, wherein the formulation is effective in restoring circadian rhythm of sleep.

Embodiment 45: A method of promoting sleep quality in a subject, the method comprising: a) identifying a subject as having a menopause-related sleep disorder; b) administering to the subject an effective amount of a formulation comprising two or more phytoestrogen compounds or analogues thereof that selectively bind to estrogen receptor beta and cross the blood-brain barrier; wherein the amount of the formulation is effective in treating the menopause-related sleep disorder.

Embodiment 46: The method of embodiment 45, wherein the subject is identified as having a menopause-related sleep disorder via actigraphy or a sleep questionnaire.

Embodiment 47: The method of embodiment 46, wherein the sleep questionnaire comprises the Pittsburgh Quality Sleep Index.

Embodiment 48: The method of embodiment 45, wherein the menopause-related sleep disorder results from vasomotor menopausal symptoms.

Embodiment 49: The method of embodiment 45, wherein the menopause-related sleep disorder does not result from vasomotor menopausal symptoms.

Embodiment 50: The method of any one of embodiments 45-49 wherein promoting sleep quality comprises increasing sleep efficiency, decreasing sleep latency, increasing sleep duration, decreasing the number of awakenings, or a combination thereof.

Embodiment 51: The method of any one of embodiments 45-50, wherein one of the phytoestrogen compounds is S-equol.

Embodiment 52: The method of any one of embodiments 45-51, wherein the two or more phytoestrogen compounds comprise genistein, daidzein, S-equol, or combinations thereof.

Embodiment 53: The method of any one of embodiments 45-52, wherein the formulation further comprises a pharmaceutically acceptable excipient or carrier.

Embodiment 54: The method of any one of embodiments 45-53, wherein the formulation is administered orally.

Embodiment 55: The method of any one of embodiments 45-53, wherein the formulation is administered topically or transdermally.

Embodiment 56: The method of any one of embodiments 45-53, wherein the formulation is administered sublingually or buccally.

Embodiment 57: The method of any one of embodiments 45-56, wherein the formulation is administered in an effective amount from about 25 mg to 75 mg.

Embodiment 58: The method of any one of embodiments 45-57, wherein the formulation is administered in an effective amount from about 50 mg to 75 mg.

Embodiment 59: The method of any one of embodiments 45-58, wherein the formulation is administered in an effective amount from about 50 mg.

Embodiment 60: The method of any one of embodiments 45-59, wherein the formulation is administered daily.

Embodiment 61: The method of any one of embodiments 45-60, wherein the formulation is effective in restoring circadian rhythm of sleep.

Embodiment 62: The method of any one of embodiments 45-60, wherein promoting sleep quality improves cognitive function.

Embodiment 63: The method of any one of embodiments 45-60, wherein promoting sleep quality improves mood symptoms.

As Used Herein, the Term “about” Refers to Plus or Minus 10% of the Referenced Number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of promoting sleep quality in a subject, the method comprising administering to the subject in need thereof an effective amount of a formulation comprising two or more phytoestrogen compounds or analogues thereof that selectively bind to estrogen receptor beta and cross the blood-brain barrier.

2. The method of claim 1, wherein the subject is a menopausal or post-menopausal woman.

3. The method of claim 1 wherein the subject has been identified as a poor sleeper.

4. The method of claim 3, wherein actigraphy or a sleep questionnaire has been used to identify the subject as a poor sleeper.

5. The method of claim 1 wherein the subject has been identified as having a menopause-related sleep disorder.

6. The method of claim 5, wherein actigraphy or a sleep questionnaire has been used to identify the menopause-related sleep disorder.

7. The method of claim 4 wherein the sleep questionnaire comprises the Pittsburgh Quality Sleep Index.

8. The method of claim 1, wherein the subject has self-identified as a poor sleeper.

9. The method of claim 1, wherein promoting sleep quality comprises increasing sleep efficiency, decreasing sleep latency, increasing sleep duration, decreasing the number of awakenings, or a combination thereof.

10. The method of claim 1, wherein one of the phytoestrogen compounds is S-equol.

11. The method of claim 1, wherein the two or more phytoestrogen compounds comprise genistein, daidzein, S-equol, or combinations thereof.

12. The method of claim 1, wherein the formulation further comprises a pharmaceutically acceptable excipient or carrier.

13. The method of claim 1, wherein the formulation is administered orally.

14. The method of claim 1, wherein the formulation is administered topically or transdermally.

15. The method of claim 1, wherein the formulation is administered sublingually or buccally.

16. The method of claim 1, wherein the formulation is administered in an effective amount from about 25 mg to 75 mg.

17. The method of claim 1, wherein the formulation is administered in an effective amount from about 50 mg to 75 mg.

18. The method of claim 1, wherein the formulation is administered in an effective amount of about 50 mg.

19. The method of claim 1, wherein the formulation is administered daily.

20. The method of claim 1, wherein the formulation is effective in restoring circadian rhythm of sleep.

21.-63. (canceled)
